(19) United States
(12) Patent Application Publication
(10) Pub. No.: US 2021/0020285 A1
(43) Pub. Date: Jan. 21, 2021

(54) BLOCKCHAIN AND CLOUD-BASED HEALTHCARE MANAGEMENT (EXPERT) SYSTEM

(71) Applicant: Michael James Hall, Miami Beach, FL (US)

(72) Inventor: Michael James Hall, Miami Beach, FL (US)

(21) Appl. No.: 16/930,318

(22) Filed: Jul. 16, 2020

Related U.S. Application Data
(60) Provisional application No. 62/874,741, filed on Jul. 16, 2019.

(51) Int. Cl. G16H 15/00; G06F 16/27; G16H 50/70; G16H 50/30

(52) U.S. Cl. CPC G16H 15/00; G16H 50/30; G16H 50/70; G06F 16/27

(57) ABSTRACT

A cloud-based system/expert system and method to provide healthcare management utilizing blockchain are disclosed. The system comprises a computing device including a processor and a memory in communication with the processor, a database in communication with the computing device, and a user device associated with a user. The computing device is configured to collect healthcare information of the user. The computing device further configured to analyze and assimilate the collected healthcare information. The analyzed healthcare information is compared with the optimal and average health metrics of same sex, race, and demography, etc. Upon comparison, the system generates a healthcare report of the user in a format understandable to a non-medically trained person. Further, the system provides suggestions and recommendations in the health report to improve the current health status of the user.

### CROSS-REFERENCE TO RELATED APPLICATION

This application claims priority to US Patent Application No. 62/874,741, titled “ BLOCKCHAIN AND CLOUD-BASED HEALTHCARE MANAGEMENT (EXPERT) SYSTEM” filed on Jul. 16, 2019. The specification of the above referenced patent application is incorporated herein by reference in its entirety.

### BACKGROUND OF THE INVENTION

### A. Technical Field

The present invention generally relates to a healthcare management system. More specifically, the present invention relates to a blockchain-based healthcare management system and method for analyzing healthcare information in real-time and enhancing the health of a user to an optimal level using a proprietary expert system.

### B. Description of Related Art

In today's digital world, different systems interact with each other for data and information exchange. As technology evolves, and artificial intelligence and machine learning become more acceptable in various fields. A health care expert system (A computer system that emulates the decision-making ability of a human expert) is one of the most important fields, which is designed to solve complex problems by reasoning through bodies of knowledge, represented mainly as if-then rules rather than through conventional procedural code. Further, it is expected that each interaction/transaction between a plurality of systems should be more secure and reliable. Currently, many systems use blockchain technology. However, blockchain technology is notoriously expensive and slow. The security of the blockchain depends on its design and could not achieve automatically by virtue of being a blockchain. Today blockchain is finding applications in every field like finance, healthcare, economics, legal, etc. Some examples from healthcare sector use cases where blockchain can be used are EHRs, drug traceability from manufacturer to consumer, clinical trials to eliminate fraudulent data modifications and interoperability, and availability of healthcare data to patients.

Further, conventional health care has increasingly relied upon trained healthcare personnel. This was in part due to the limited availability of education, and due to the limited access to medical knowledge bases used by health care. Patients had to rely on limited medical resources. Hence, patients typically have not had easy access to their healthcare information and records. This makes the patient lost their trust in the healthcare personnel. Few existing patent applications attempted to address the aforementioned problems are discussed as follows.

US20170039330A1 of Theodore Tanner, et.al., entitled “System and method for decentralized autonomous healthcare economy platform” discloses a system and method to provide a decentralized autonomous healthcare economy platform. The system aggregates the healthcare data from a plurality of health data sources for real-time analysis of the information. The system further processes the data via a hybrid federated and peer-to-peer distributed processing architectures, and provides a health score and alerts the user based on the analyzed data.

US20180165416A1 of Manoj Saxena, et.al., entitled “method for providing healthcare-related, blockchain-associated cognitive insights using blockchains”. The method for providing healthcare-related, blockchain-associated cognitive insights comprising: receiving data from a plurality of data sources, at least some of the plurality of data sources comprising healthcare-related data sources and blockchain data sources; processing the data from the plurality of data sources to provide a healthcare-related, blockchain-associated cognitive insight; and, providing the healthcare-related, blockchain-associated cognitive insight to a destination.

Even though health information is provided to the user, it is difficult to determine exactly the health status without proper user interface and format. Further non-professionals without medical knowledge, still require training or assistance of health care professionals for health care advice.

Therefore, there exists a need for an improved healthcare management system to analyze and monitor the health of the user. Further, there is a need for a system to continuously monitor and assist the users to improve their health. Also, there is a need for a system to provide healthcare information in a format understandable by a non-medically trained person.

### SUMMARY OF THE INVENTION

The present invention discloses a cloud-based healthcare management system and method utilizing blockchain to continuously monitor and assist the users to improve their health. Further, the system provides healthcare information in a format understandable by a non-medically trained person.

The system is configured to analyze the healthcare information in real-time and enhance the health of a user to an optimum level. According to the present invention, the system comprises a computing device, a database in communication with the computing device, and a user device in communication with the computing device. The computing device comprises a processor and a memory in communication with the processor. The memory stores a set of instructions executable by the processor. The user device associated with the user is configured to access the computing device via a network. In one embodiment, the computing device is a server.

In one embodiment, the computing device is configured to collect and assimilate a plurality of healthcare information of the user. The computing device further configured to analyze the assimilated healthcare information of the user. The computing device further configured to compare the healthcare information of the user with the optional and average health metrics of same sex, race, and demography. The computing device further configured to generate a healthcare report of the user in a format understandable to a non-medically trained person.

In one embodiment, the healthcare report includes a graphical representation of the user's healthcare information. The graphical representation includes, but not limited to a spider web chart, pie chart, etc. In an exemplary embodiment, the health data of the patient could be represented with a spider web chart. The spider web chart represents different health categories, for example, the brain, heart, lungs, thyroid, etc. of the user or patient based on the available patient's health data. The healthcare report includes one or more suggestions and recommendations to improve the health of the user. In one embodiment, the healthcare report further includes a score, which represents the health status of the user. In one embodiment, the score is generated based on multiple criteria and comparisons performed by the healthcare management system.

In one embodiment, the healthcare information provided by the system includes a dashboard comprising the graphical representation of the user's healthcare information. In one embodiment, the computing device is configured to timestamps every transaction that is non-erasable during the analysis of healthcare information. The system utilizes one or more peer-to-peer networks and a plurality of nodes to analyze the healthcare information. In one embodiment, the peer-to-peer network is configured to enable users/patients, doctors/physical trainers, and third parties for securely sharing and collecting the protected health information (PHI) among a plurality of nodes. In one embodiment, the plurality of nodes interacts/transmits healthcare data among them via a blockchain network. In one embodiment, the nodes could be, but not limited to, computers, laptops, tablets, and smartphones. Further, a log-management in the system helps in easy auditing and in compliance with issues in the healthcare information.

In one embodiment, a method for generating the healthcare report based on the healthcare information of a user or a patient or an individual comprising a computing device includes a processor and a memory in communication with the processor, wherein the memory stores a set of instructions executable by the processor, a database in communication with the computing device, and a user device associated with the user configured to access the computing device via a network. The method comprises the step of collecting a plurality of healthcare information of the user. In one embodiment, the collected information includes, but not limited to subjective information such as personal details, and objective information such as disease details, treatment undertaking, statistics report, medical records history, imaginary and multi-media reports such as x-ray or scan reports and/or matrices collected over time. The healthcare system views and manages the collected healthcare information in real-time. In one embodiment, the healthcare system is a distributed blockchain-based healthcare system. In some embodiments, the healthcare system could utilize a centralized database for managing healthcare information.

The method further comprises a step of analyzing and assimilating the collected healthcare information of the user via the blockchain-based healthcare system. The method further comprises a step of comparing the analyzed and assimilated healthcare information of the user with the optimal and average health metrics of same sex, race, and demography. The method further comprises a step of timestamping of every transaction that is non-erasable during the analysis of healthcare information. The method further comprises a step of generating a healthcare report of the user in a format understandable by a non-medically trained person or a person without a knowledge relating to the medical field. Also, the system provides suggestions or comments to the user to improve their health.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and structures disclosed herein. The description of a method step or a structure referenced by a numeral in a drawing is applicable to the description of that method step or structure shown by that same numeral in any subsequent drawing herein.

FIG. 1 exemplarily illustrates an environment of a cloud-based healthcare management system utilizing blockchain, according to an embodiment of the present invention.

FIG. 2 exemplarily illustrates a flowchart of a method for generating a healthcare report, according to an embodiment of the present invention.

FIG. 3 exemplarily illustrates a screenshot of a user interface for accessing one or more features, according to an embodiment of the present invention.

FIG. 4 exemplarily illustrates a screenshot of a user interface for managing health information of the user, according to an embodiment of the present invention.

FIG. 5 exemplarily illustrates a detailed view of a user dashboard, according to an embodiment of the present invention

FIG. 6 exemplarily illustrates a screenshot of the user dashboard, according to an embodiment of the present invention.

FIG. 7 exemplarily illustrates a screenshot of a graphical representation of a patient's health report, according to an embodiment of the present invention.

FIG. 8 exemplarily illustrates a screenshot of the graphical representation of a patient's health report with suggestions to enhance the health of the user to an optimal level, according to an embodiment of the present invention.

### DETAILED DESCRIPTION OF EMBODIMENTS

A description of embodiments of the present invention will now be given with reference to the Figures. It is expected that the present invention may be embodied in other specific forms without departing from its spirit or essential characteristics. The described embodiments are to be considered in all respects only as illustrative and not restrictive.

A description of embodiments of the present invention will now be given with reference to the Figures. It is expected that the present invention may be embodied in other specific forms without departing from its spirit or essential characteristics. The described embodiments are to be considered in all respects only as illustrative and not restrictive.

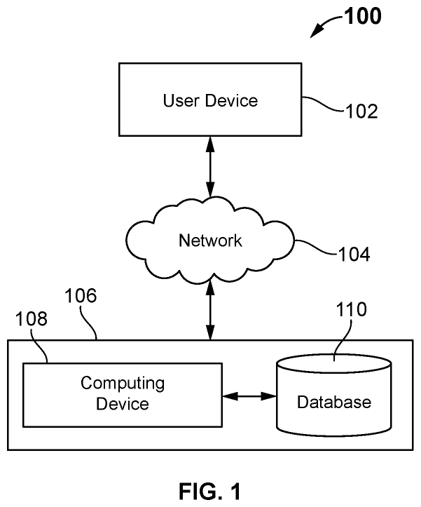

Referring to FIG. 1, a block diagram of a system implemented in a computer-implemented environment 100, according to an embodiment of the present invention. The computer-implemented environment 100 comprises a user device 102, a network 104, and a healthcare management system 106. In an embodiment, the healthcare management system 106 comprises a computing device 108 and at least one database 110. In one embodiment, the computing device 108 is a server. In an embodiment, the user device 102 is enabled to access the healthcare management system 106 via the network 104. In one embodiment, the healthcare management system is a distributed blockchain-based healthcare system. In one embodiment, the user device 102 is at least any one a desktop, a laptop, a tablet, a mobile phone, a smartphone, and /or other suitable handheld electronic communication devices. In an embodiment, the network 104 could be Wi-Fi, WiMAX, and wireless local area network (WLAN). The blockchain is a decentralized, distributed and public digital ledger that is used to record transactions across many computers so that any involved record cannot be altered retroactively, without the alteration of all subsequent blocks. This allows the participants to verify the transactions independently and relatively inexpensively. In one embodiment, the blockchain system is managed autonomously using a peer-to-peer network and a distributed timestamping server.

In one embodiment, the computing device 108 comprises a processor and a memory in communication with the processor. The memory stores a set of instructions executable by the processor. In one embodiment, the computing device 108 is at least one of a general or special purpose computer. The computing device 108 could be operated as a single computer, which can be a hardware and/or software server, a workstation, a desktop, a laptop, a tablet, a mobile phone, a mainframe, a supercomputer, a server farm, and so forth. In some embodiments, the computer could be a touchscreen and/or a non-touchscreen and adopted to run on any type of OS, such as iOS™, Windows™, Android™, Unix™, Linux™ and/or others. In one embodiment, the plurality of computers is in communication with each other, via networks. Such communication can be via a software application, a mobile app, a browser, an OS, and/or any combination thereof.

The computing device 108 is configured to collect healthcare information of the user. The computing device 108 is further configured to generate a healthcare report based on the collected healthcare information. In one embodiment, the database 110 is accessible by the computing device 108. In another embodiment, the database 110 is integrated into the computing device 108 or separate from it. In some embodiments, the database 110 resides in a connected server or in a cloud computing service. Regardless of location, the database 110 comprises a memory to store and organize certain data for use by the computing device 108.

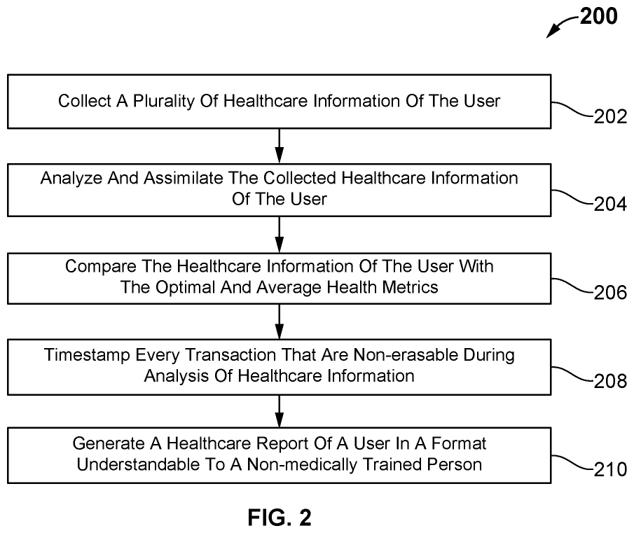

Referring to FIG. 2, a flowchart of a method 200 for generating the healthcare report based on the healthcare information of a user or a patient or an individual comprising a computing device includes a processor and a memory in communication with the processor, wherein the memory stores a set of instructions executable by the processor, a database in communication with the computing device, and a user device associated with the user configured to access the computing device via a network. The system collects, assimilates, and visually represents the key health-related information to manage and encourage the patient for positive physiological and psychological health change. The system provides an improved method for measuring the health information and generates a complete overview of the patient's current health status based on a plurality of anatomical and physiological metrics.

The method 200 comprises the step 202 of collecting a plurality of healthcare information of the user. In one embodiment, the collected information includes, but not limited to subjective information such as personal details, and objective information such as disease details, treatment undertaking, statistics report, medical records history, imaginary and multi-media reports such as x-ray or scan reports and/or matrices collected over time. The healthcare system views and manages the collected healthcare information in real-time. In one embodiment, the healthcare system is a distributed blockchain-based healthcare system. In some embodiments, the healthcare system could utilize a centralized database for managing healthcare information.

The method 200 further comprises a step 204 of analyzing and assimilating the collected healthcare information of the user via the blockchain-based healthcare system. the method 200 further comprises a step 206 of comparing the analyzed and assimilated healthcare information of the user with the optimal and average health metrics of same sex, race, and demography. The method 200 further comprises a step 208 of timestamping of every transaction that is non-erasable during the analysis of healthcare information. The method 200 further comprises a step 210 of generating a healthcare report of the user in a format understandable by a non-medically trained person or a person without a knowledge relating to the medical field. Also, the system provides suggestions or comments to the user to improve their health. The system utilizes one or more peer-to-peer networks and a number of nodes for processing the healthcare information. In one embodiment, the peer-to-peer network is configured to enable users/patients, doctors/physical trainers, and third parties for securely sharing and collecting the protected health information (PHI) among a plurality of nodes. In one embodiment, the plurality of nodes interacts/transmits healthcare data among them via a blockchain network. In one embodiment, the nodes could be, but not limited to, computers, laptops, tablets, and smartphones. In one embodiment, the system further includes a log-management for easy auditing and in compliance issues relating to healthcare information.

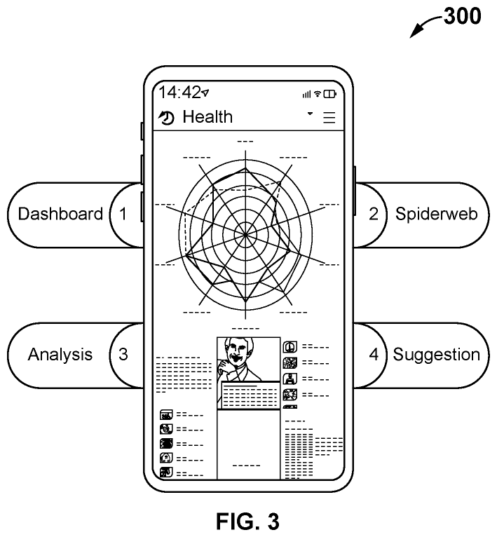

Referring to FIG. 3, a screenshot 300 of a user interface for accessing one or more features is disclosed, according to an embodiment of the present invention. In one embodiment, the features include a dashboard, spiderweb, analysis, and suggestion. The screenshot 300 shows the user interface for a mobile device. In one embodiment, the dashboard provides alerts to some targeted network vulnerabilities such as Advanced Malware, Advanced Persistent Threat (APT). The customizable dashboard makes the system more user-friendly. In one embodiment, the spiderweb provides the current health status of the patient. In some embodiment, the health status could be represented in other types of graphical representation include, but not limited to, pie chart, bar chart, or sunburst.

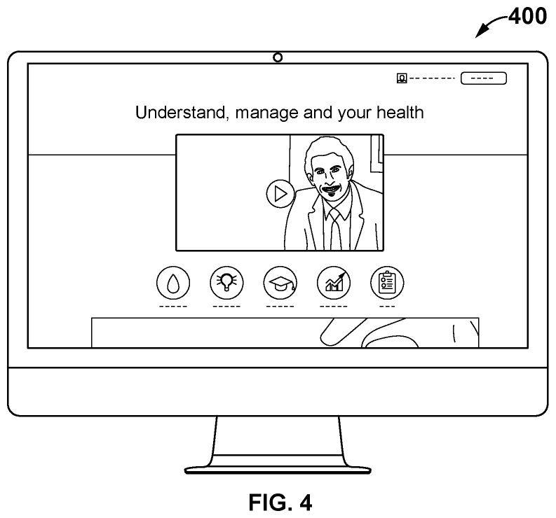

FIGS. 4 to 8 explain the features of blockchain-based healthcare management system in a web view, according to an embodiment of the present invention. A screenshot 400 of a user interface for managing health information of the user is shown in FIG. 4. In one embodiment, the screenshot 400 is a home page, which enables the user to register or sign up on the website. In one embodiment, the screenshot 400 further comprises one or more features such as to measure, understand, optimizes, and track the procedures.

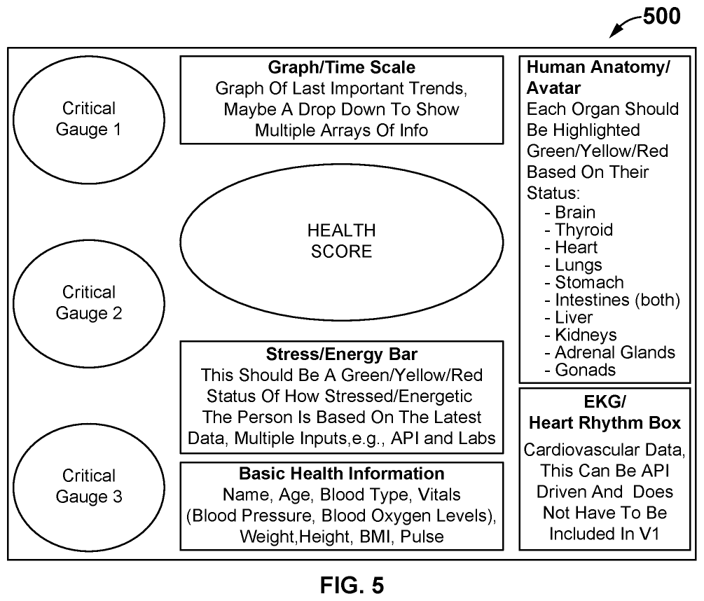

Referring to FIG. 5, a detailed view of a user dashboard 500 is disclosed, according to an embodiment of the present invention. The blockchain-based algorithm performs three-different stages of operation to help the user to manage their health information. In one embodiment, the three-stage of operation includes, but not limited to, collect, assimilate, and visual representation of key health-related data points in the user dashboard. The three stages are represented as critical gauge 1, critical gauge 2, and critical gauge 3, respectively. In one embodiment, the system collects user's health-related data based on unique evidence-based anatomical and physiological metrics using blockchain-based algorithm. In one embodiment, the health data could be a human anatomy/avatar collected from each organ of the user such as the brain, thyroid, heart, lungs, stomach, intestines (small intestine and large intestine), liver, kidneys, adrenal glands, and gonads.

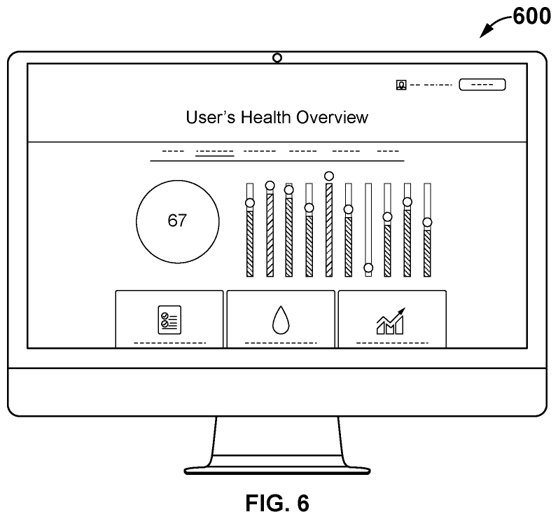

In one embodiment, the blockchain-based algorithm provides a complete overview of the user's health condition based on the collected data as shown in FIG. 6. The overview of the user's health condition helps the user to manage their health information in real-time and encourage positive psychological and behavioral changes of the user. Further, the system collects EKG or ECG (Electrocardiogram)/heart rhythm box include, but not limited to, cardiovascular data, which is an API driven data (Application Programming Interface). In one embodiment, the system uses API to calculate the health information and create a health value using the health data of 10 main areas such as the brain, neurosensory, hormone, cardiovascular, digestive, dermatologic, immunologic, musculoskeletal, infectious, genetic and environmental health. In one embodiment, the dashboard 500 comprises a stress bar/energy bar represented in different color variations, for example, green/yellow/red status bar. The different color stress bar shows the stressed/energetic status of the user or patient. Further, the system collects some basic health information of the user such as name, age, blood type, vitals (blood pressure and blood oxygen levels), weight, height, BMI, and pulse. Also, the collected health information is used for future analysis and report generation.

In one embodiment, the system compares each aspect of the health data with the optimal and average health metrics within the same sex, race, and demographics. Based on the result, the user receives a number value/score and a health graph. In one embodiment, the health graph could be a spider web chart. Further, the health graph targets one or more key area of weakness and make customized suggestions to improve health in order to reach optimal health. Referring to FIG. 6, a screenshot 600 of a user dashboard is disclosed, according to an embodiment of the present invention. The dashboard enables the user to view their health report, general health tips, and tips to improve the current health situation. In one embodiment, the dashboard 600 comprises a set of stress bars/energy bars represented in different color variations to show the stressed/energetic status of the user or patient.

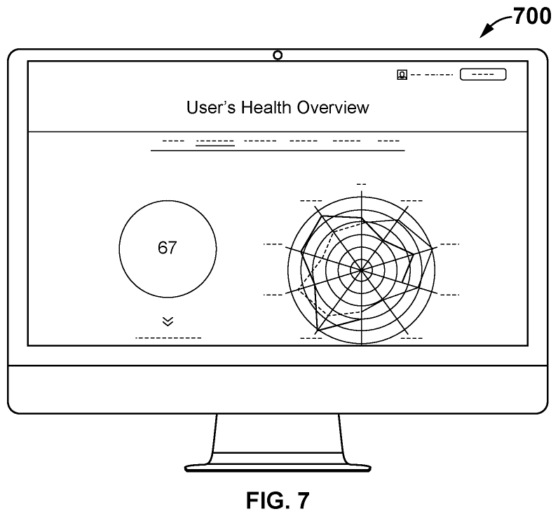

FIG. 7 exemplarily illustrates a screenshot 700 of graphical representation of a patient's health report, according to an embodiment of the present invention. The graphical representation includes, but not limited to a spider web chart, pie chart, etc. In an exemplary embodiment, the health data of the patient could be represented with a spider web chart. The spider web chart represents different health categories, for example, the brain, heart, lungs, thyroid, etc. of the user or patient based on the available patient's health data. The screenshot 700 provides an easy and in-depth analysis of the patient's health based on their health information in the database. During analysis, the system performs the following steps comprising: deconstructing the collected data in the database into blockchain code, storing the deconstructed data with 256-bit encryption, and analyzing the encrypted data with the blockchain-based algorithm. The analyzed healthcare information is compared with the optimal and average health metrics within same sex, race, and demographics. Based on the analysis, the system generates the health situation report, a graph, and/or a health web, which highlights key areas of strengths and weaknesses to improve the current health situation.

In one embodiment, the graphical representation of health report contains a plurality of health metrics such as the brain, neurosensory, musculoskeletal, hormones, cardiovascular, digestive, dermatologic, immunologic, genetics, and environment. The details of the brain include cognitive, proprioceptive, and psychological aspects. The neurology includes vision, hearing, scent, taste, and touch sensation. The musculoskeletal includes bone composition, tendon, and skeletal resistance. The hormones include sex, metabolic and stress level. The digestive includes vitamin and minerals, enzyme level, and bowel health details. The dermatologic includes chrono-aging and photo-aging, and the immunologic includes chronic parasite, viral, autoimmune, and nutritional antigens. The genetic metric includes gestation, DNA length, and intracellular antioxidant levels. The environment includes the details of metal and air toxicity. In one embodiment, the system assigns a value to the health report, where the value is generated based on the multiple criteria and comparisons performed during analysis. In one embodiment, the assigned value is a healthy FICO score.

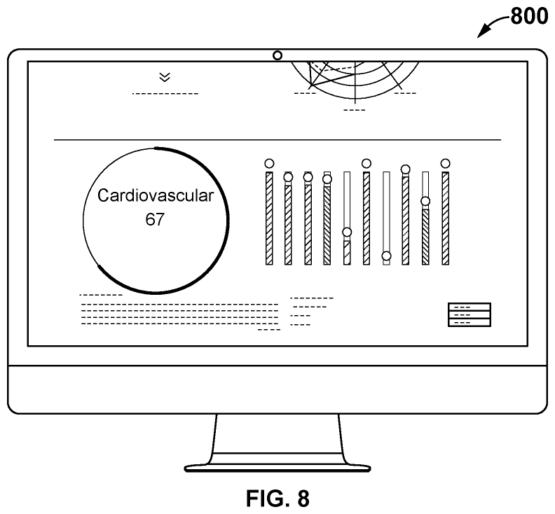

Referring to FIG. 8, a screenshot 800 of graphical representation of a patient's health report with suggestions to enhance the health of the user to an optimal level, according to an embodiment of the present invention. In one embodiment, the system provides customized comments or suggestions to improve health in order to reach optimal health. The medical information of each health area and their suggestions are provided along with a score of about 1 to 100 in each test result. In one embodiment, the health area includes, but not limited to, brain, neurosensory, musculoskeletal, hormones, cardiovascular, digestive, dermatologic, immunologic, genetics, and environment. The health report is sent to the user with suggestions and recommendations to improve their health. Again, the system starts processing new data with dashboard feedback for the user.

The system is in-built in a secured platform such as blockchain technology based on 256-bit encryption. The system enables the end-user to optimize their healthcare outcomes and costs. Also, the system enables secured data transfer over vast distances in time and GDPR compliant. The healthcare information could be accessed at any time from any place by the registered users. The users include, but not limited to, a registered patient or individual, a doctor, and an authorized third parties. Also, the system meets all federal and state guidelines to avoid harm within or outside the system. The information further utilized to make the most accurate statistics and overviews. The healthcare information could be accessed on an individual basis and segregated to ensure that the individual only could view their personalized data. The system uses Protected Health Information (PHI) gathered from the physician or doctor for generating healthcare report. The system uses accurate anonymous medical statistics to improve overall health issues.

The advantages and applications of the present invention include, but not limited to the following: The blockchain-based healthcare management system could be utilized by healthcare providers, sports teams, fortune 500 companies, military, and other fields that requires healthcare management. The health care providers of any size could offer the service of the present invention for their clients or patients in order to improve the understanding of patients and outlook towards their health. The clinics could offer easy-to-understand suggestions to improve the individual's health and offer potential treatments. The system is also helpful to optimize the performance of professional sports teams. The system aids the companies to optimize the personal health of an employee, which reduces the insurance cost and increases productivity and revenue. In addition, the system also utilized in military applications. Detecting and predicting susceptibility to infections or nutritional deficiencies with the system improves the health of an individual soldier and the mission.

Preferred embodiments of this invention are described herein, including the best mode known to the inventors for carrying out the invention. It should be understood that the illustrated embodiments are exemplary only and should not be taken as limiting the scope of the invention.

The foregoing description comprise illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings in the foregoing descriptions. Although specific terms may be employed herein, they are used only in generic and descriptive sense and not for purposes of limitation. Accordingly, the present invention is not limited to the specific embodiments illustrated herein.

1. A blockchain-based computer-implemented healthcare management system, comprising:
a computing device including a processor and a memory in communication with the processor, wherein the memory stores a set of instructions executable by the processor;
a database in communication with the computing device stores healthcare information of a plurality of users, and
a user device associated with a user configured to access the computing device via a network wherein the computing device is configured to:
collect a plurality of healthcare information of the user,
analyze and assimilate the collected healthcare information of the user,
compare the healthcare information of the user with the optimal and average health metrics of same sex, race, and demography, and
generate a healthcare report of the user in a format understandable to a non-medically trained person.

2. The system of claim 1, generates a complete overview of the patient's current health status based on a plurality of anatomical and physiological metrics.

3. The system of claim 1, further utilizes an application program interface (API) to calculate the health information and create a health value using the health data of main areas including the brain, neurosensory, hormone, cardiovascular, digestive, dermatologic, immunologic, musculoskeletal, infectious, genetic and environmental health.

4. The system of claim 1, wherein the healthcare report includes a graphical representation of health information.

5. The system of claim 1, wherein the graphical representation is a spider web chart, wherein the spider web chart represents one or more health-related categories of the user or patient.

6. The system of claim 1, wherein the healthcare report includes suggestion data to improve the health of the user.

7. The system of claim 1, wherein the healthcare report further includes a score representing the health status of the user.

8. The system of claim 1, wherein the healthcare report includes a dashboard comprising the graphical representation of health information.

9. The system of claim 1, wherein the computing device is configured to timestamps every transaction that is non-erasable during the analysis of healthcare information.

10. A blockchain-based computer-implemented method of generating a healthcare report using multiple healthcare information of a user, comprising a computing device including a processor and a memory in communication with the processor, wherein the memory stores a set of instructions executable by the processor, a database in communication with the computing device stores healthcare information of a plurality of users, and a user device associated with a user configured to access the computing device via a network,
wherein the method comprises the steps of:
collecting a plurality of healthcare information of the user;
analyzing and assimilating the collected healthcare information of the user;
comparing the healthcare information of the user with the optimal and average health metrics of same sex, race, and demography, and
generating a healthcare report of the user in a format understandable to a non-medically trained person.

11. The method of claim 10, generates a complete overview of the patient's current health status based on a plurality of anatomical and physiological metrics.

12. The method of claim 10, further utilizes an application program interface (API) to calculate the health information and create a health value using the health data of main areas including the brain, neurosensory, hormone, cardiovascular, digestive, dermatologic, immunologic, musculoskeletal, infectious, genetic and environmental health.

13. The method of claim 10, wherein the healthcare report includes a graphical representation of health information.

14. The method of claim 10, wherein the graphical representation is a spider web chart, wherein the spider web chart represents one or more health-related categories of the user or patient.

15. The method of claim 10, wherein the healthcare report includes suggestion data to improve the health of the user.

16. The method of claim 10, wherein the healthcare report further includes a score representing the health status of the user.

17. The method of claim 10, wherein the healthcare report includes a dashboard comprising the graphical representation of health information.

18. The method of claim 10, wherein the computing device is configured to timestamp every transaction that is non-erasable during the analysis of healthcare information.
